(19) United States
(12) Patent Application Publication
(10) Pub. No.: US 2025/0249410 A1
(43) Pub. Date: Aug. 7, 2025

(54) HIGHLY SELECTIVE ACID-RESISTANT POLYAMIDE NANOFILTRATION MEMBRANE CONTAINING TROGER'S BASE AND PREPARATION METHOD THEREOF

(71) Applicant: Zhejiang University of Technology, Hangzhou (CN)

(72) Inventors: Lifen LIU, Hangzhou (CN); Liuyang GONG, Hangzhou (CN); Nian ZHANG, Hangzhou (CN); Beina DONG, Hangzhou (CN); Li XIA, Hangzhou (CN)

(73) Assignee: Zhejiang University of Technology, Hangzhou (CN)

(21) Appl. No.: 19/023,282

(22) Filed: Jan. 16, 2025

(30) Foreign Application Priority Data
Feb. 6, 2024 (CN) 202410168422.3

(51) Int. Cl. B01D 67/00; B01D 61/02; B01D 69/02; B01D 69/10; B01D 71/56; C02F 1/44; C07D 487/08

(52) U.S. Cl. CPC B01D 67/0006; B01D 61/027; B01D 67/0018; B01D 69/02; B01D 69/105; B01D 69/1071; B01D 71/56; C02F 1/442; C07D 487/08; B01D 2323/081; B01D 2325/30

(57) ABSTRACT

The present invention discloses a highly selective acid-resistant polyamide nanofiltration membrane containing Tröger's base and a preparation method thereof. The method comprises: preparing a Tröger's base phenylenediamine monomer, dissolving the monomer together with polyethersulfone in a polar solvent to prepare a casting solution, and then coating the casting solution on a non-woven fabric to prepare a polyethersulfone support membrane with a surface rich in PDA-TB by non-solvent induced phase separation, and finally contacting with a polyacyl chloride solution by one side to perform a polymerization reaction to prepare a highly selective acid-resistant polyamide nanofiltration membrane containing Tröger's base. Compared with the conventional poly(piperazine-amide) nanofiltration membrane, the nanofiltration membrane of the present invention has greatly improved acid resistance and higher separation selectivity.

### CROSS-REFERENCE TO RELATED APPLICATIONS

This disclosure claims priority to Chinese Patent Application No. 202410168422.3 filed in China on Feb. 6, 2024, which is hereby incorporated by reference in its entirety.

### TECHNICAL FIELD

The present disclosure relates to the technical field of polymer separation membranes for water treatment, and in particular to a highly selective acid-resistant polyamide nanofiltration membrane containing Tröger's base and a preparation method thereof.

### BACKGROUND

Over the last few decades, the global industrial production has expanded rapidly. Production processes such as chemical medicine, metallurgy, textile, electroplating and papermaking also produce a large amount of acidic wastewater while consuming a large amount of water resources. The direct discharge of these acidic wastewater will pollute the water source, destroy the ecological environment and endanger human health. Although some acids and heavy metals in wastewater can be recovered by using traditional methods such as adsorption, precipitation and extraction, the process of these methods is often complicated, inefficient and costly, and also has a risk of secondary pollution. Nanofiltration (NF) is a new type of efficient and environmentally friendly separation technology, and it can effectively separate monovalent and polyvalent salts, and also reject the organic matter with a molecular weight of 200˜1000 Da. Nanofiltration separation technology is easy to operate, energy-saving, efficient and has high separation accuracy. It has been widely used in wastewater treatment, drinking water purification, product separation and purification and other fields.

The core of the nanofiltration separation technology is a nanofiltration membrane. At present, a commercial poly(piperazine-amide) composite nanofiltration membrane commonly in the market is prepared by an interfacial polymerization reaction of piperazine and trimesoyl chloride on a polysulfone support membrane. The poly(piperazine-amide) bond is easily attacked by protons under strong acidic conditions, resulting in chemical bonds breakage. This leads to a sharp decrease in the separation performance of the poly(piperazine-amide) nanofiltration membrane, greatly affects the service life of the membrane, and seriously hinders the separation application of the nanofiltration technology under acidic conditions. Therefore, it is urgent to develop high-performance acid-resistant nanofiltration membranes from sources.

Since the stability of both sulfonyl and amine bonds and the hyperconjugation effect of triazine ring can withstand the attack of strong acids, materials such as polysulfonamide and polyamine containing the above structures have been reported in recent years to be used to develop acid-resistant nanofiltration membranes. For example, Yu Sanchuan et al. (J. Membr. Sci., 2012, 415:122-131; Desalination, 2013, 315:164-172) prepared a series of acid-resistant polysulfonamide nanofiltration membranes by interfacial polymerization of naphthalenesulfonyl chloride monomer (such as NTSC) and polyamine monomer (such as piperazine PIP, etc). Patents (such as US2016/0051944A1, CN107349804A, CN109999666A, etc) reported a series of acid-resistant polyamine nanofiltration membranes prepared by polymerization of polyamine compounds (such as PEI, diethylenetriamine or triethylenetetramine, etc.) and crosslinking agents (such as cyanuric chloride or benzyl chloride, etc). Zhang Lin et al. (J. Membr. Sci., 2018, 546:225-233) synthesized a poly(triazine)amine (TPT) precursor by the pre-reaction of cyanuric chloride (CC) and piperazine, and then successfully prepared an acid-resistant poly(triazine-amide) nanofiltration membrane by interfacial polymerization of TPT precursor with trimesoyl chloride (TMC). Although all above membranes show good stability under strong acid conditions with pH=0-1, the introduction of acid-resistant groups will reduce the reaction activity of monomers to a certain extent, thereby affecting the interfacial polymerization reaction and the separation performance of the membrane such as selectivity and water flux. When improvements are made by adding additives and catalysts, or performing multiple interfacial polymerizations, the membrane preparation process will be cumbersome, complex, inefficient and costly. The patent CN2022102595270 mixed a diamine monomer TBDA containing Tröger's base with piperazine to modify the poly(piperazine-amide) nanofiltration membrane, which was previously reported by the inventor. Although the water flux of this modified NF membrane was greatly increased, its acid resistance was only slightly improved, and it was difficult to withstand a high concentration of strong acid.

### SUMMARY

The purpose of the present disclosure is to overcome the above-mentioned deficiencies of the prior art, in particular to overcome the lack of acid resistance of the traditional poly(piperazine-amide) nanofiltration membrane or the poly(piperazine-amide) nanofiltration membrane previously prepared via TBDA doping modification by the inventor, and to provide a highly selective acid-resistant polyamide nanofiltration membrane containing Tröger's base and a preparation method thereof.

In one aspect, the present disclosure provides a method for preparing a highly selective acid-resistant polyamide nanofiltration membrane containing Tröger's base, the method comprises:
preparing a Tröger's base phenylenediamine monomer;
dispersing the Troger's base phenylenediamine monomer and the polyethersulfone into a polar solvent, stirring uniformly, and then standing for defoaming to obtain a casting solution;
coating the casting solution on a non-woven fabric to form a liquid film, and then placing it in a coagulation bath aqueous solution for a non-solvent induced phase separation, wherein the time of non-solvent induced phase separation is 30 to 120 seconds. After this, rinsing the membrane surface with deionized water, and then drying the membrane at room temperature to obtain a polyethersulfone supporting membrane with a surface rich in Tröger's base phenylenediamine monomer; and
the organic phase solution containing the polyacyl chloride monomer is poured onto the polyethersulfone support membrane with the surface rich in the Tröger's base phenylenediamine monomer. After reacting for 30 to 300 seconds, the remaining organic phase solution is removed, and then heat-treated at 25° C. to 40° C. for 20 to 60 minutes. Finally, the prepared membrane is immersed in deionized water for cleaning, and thus a highly selective acid-resistant polyamide nanofiltration membrane containing Tröger's bases is obtained.

Optionally, the step of preparing the Tröger's base phenylenediamine monomer comprises:
dissolving 2 to 12 parts by mass of a halogenated aniline monomer molecule into 100 parts by mass of an acidic solvent, then adding 2 to 6 parts by mass of an aldehyde monomer, mixing uniformly, and reacting at −10° C. to 40° C. for 12 to 96 hours to obtain a halogenated benzene intermediate containing Tröger's bases; and
dispersing 1 to 10 parts by mass of the halogenated benzene intermediate containing Tröger's bases into 20 parts by mass of an aromatic hydrocarbon monomer, and under the condition of an inert gas, 0.03 to 0.06 parts by mass of a catalyst, 0.04 to 0.08 parts by mass of 1, l′-binaphthyl-2,2′-bisdiphenylphosphine, 0.8 to 1.2 parts by mass of sodium tert-butoxide and 0.8 to 1.6 parts by mass of benzophenone imine are added, mixed uniformly and reacted at 80° C. to 120° C. for 12 to 48 hours. After this, the reaction continues for 1 to 6 hours at the presence of a mixed solvent of 4 to 16 parts by mass of tetrahydrofuran and 10 to 40 parts by mass of 1 to 3 mol/L inorganic acid, and finally the Tröger's base phenylenediamine monomer is obtained.

Optionally, the halogenated aniline monomer molecule is one or more selected from 4-fluoroaniline, 4-bromoaniline, 4-iodoaniline and 4-chloroaniline.

Optionally, the aldehyde monomer is one or more selected from formaldehyde, paraformaldehyde, benzaldehyde, phenylacetaldehyde, phenylpropionaldehyde and octanal.

Optionally, the aromatic hydrocarbon monomer is one or more selected from benzene, toluene, xylene and ethylbenzene.

Optionally, the inert gas is at least one selected from nitrogen and argon; and the inorganic acid is one selected from hydrochloric acid, sulfuric acid and nitric acid.

Optionally, the catalyst is tris(dibenzylideneacetone) dipalladium.

Optionally, the stirring is performed at a temperature of 20° C. to 60° C. for 12 to 30 hours, and then the defoaming is performed by standing for 12 to 24 hours.

Optionally, the weight percentages of the Tröger's base phenylenediamine monomer, the polyethersulfone and the polar solvent are calculated as 100% by weight:

| | |
|---|---|
| Tröger's base diamine monomer | 1 wt % to 3 wt %; |
| polyethersulfone | 10 wt % to 20 wt %; |
| polar solvent | balance. |

Optionally, the polar solvent is one or more selected from N,N-dimethylformamide, N,N-dimethylacetamide or dimethyl sulfoxide, and N-methylpyrrolidone.

Optionally, the volume of the coagulation bath aqueous solution is 2 to 4 L, and the time of the non-solvent induced phase separation is 30 to 120 seconds.

Optionally, the time of drying at room temperature is 10 to 30 minutes.

Optionally, the polyacyl chloride monomer is one or more selected from isophthaloyl chloride, 1,3,5-triazine-2,4,6-triacyl chloride, adipoyl chloride, 4-biphenylcarbonyl chloride, trimesoyl chloride and phthaloyl chloride.

Optionally, the concentration of the polyacyl chloride monomer in the organic phase solution containing the polyacyl chloride monomer is 0.01 wt % to 0.2 wt %; the organic solvent in the organic phase solution containing the polyacyl chloride monomer is one or more selected from Isopar G, cyclohexane, n-alkane, n-hexane and n-heptane.

In another aspect, the present disclosure provides a highly selective acid-resistant polyamide nanofiltration membrane containing Tröger's base prepared by the above method.

### BRIEF DESCRIPTION OF THE DRAWINGS

FIG. 1 is a structural diagram of a functional monomer of Tröger's base phenylenediamine (PDA-TB) described in the present disclosure.

FIG. 2 is a Fourier attenuation total reflection infrared spectrum (ATR-FTIR) diagram of the highly selective acid-resistant polyamide nanofiltration membrane containing Tröger's base (M1) prepared in Example 1 of the present disclosure, the poly(piperazine-amide) nanofiltration membrane (M2) prepared in Comparative Example 1 of the present disclosure, and the blank PES porous support membrane.

FIG. 3 shows a SEM image of the highly selective acid-resistant polyamide nanofiltration membrane containing Tröger's base (a) prepared in Example 1 of the present disclosure before and after long-term immersion in a 20 wt % H₂SO₄ solution for 30 days, and a SEM image of the polypiperazineamide nanofiltration membrane (b) prepared in Comparative Example 1 of the present disclosure before and after long-term immersion in a 20 wt % H₂SO₄ solution for 12 days.

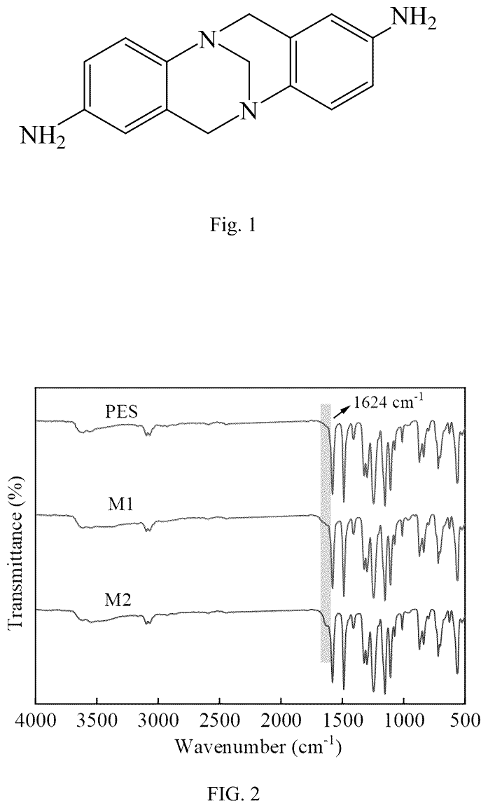

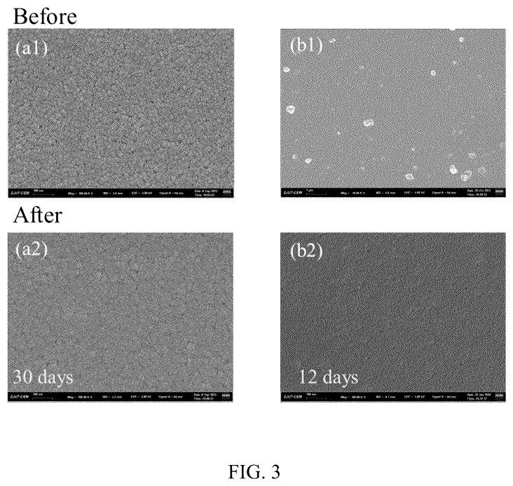

### DETAILED DESCRIPTION

The present disclosure will be further described in detail below with reference to specific embodiments, but the content and scope of the invention of this patent are not limited to the following embodiments, and all changes or improved embodiments should be included within the scope of the present invention without departing from the content and scope of the present invention.

The purpose of the present disclosure is to overcome the deficiencies in the prior art, in particular to overcome the lack of acid resistance of the traditional poly(piperazine-amide) nanofiltration membrane or the poly(piperazine-amide) nanofiltration membrane previously prepared by TBDA doping modification by the inventor, and to provide a highly selective acid-resistant polyamide nanofiltration membrane containing Tröger's base and a preparation method thereof. Specifically, the nanofiltration membrane is prepared by the following method: a single Tröger's base phenylenediamine monomer (PDA-TB) and polyethersulfone are made into a casting solution, which is coated on a non-woven fabric to form a liquid film, then this film is placed in a coagulation bath aqueous solution for non-solvent induced phase separation to obtain a polyethersulfone porous support membrane with a surface rich in PDA-TB. After cleaning the surface with deionized water, the membrane is contacted with an organic phase solution containing an acyl chloride monomer on one side for interfacial polymerization, and finally heat treatment is performed. Since the PDA-TB molecule contains a Tröger's base with a unique rigid twisted structure, the nanofiltration membrane has excellent acid resistance and narrower pore size distribution, thereby improving the separation selectivity of the nanofiltration membrane.

The present disclosure also provides a method for synthesizing a Tröger's base phenylenediamine monomer. The Tröger's base phenylenediamine monomer prepared by this method is introduced to the surface of the polyethersulfone support layer by a non-solvent induced phase separation method, and then a highly selective acid-resistant polyamide nanofiltration membrane containing Tröger's base is prepared by interfacial polymerization.

Specifically, the method for synthesizing the Tröger's base phenylenediamine monomer includes the following steps:
dissolving 2 to 12 parts by mass of a halogenated aniline monomer molecule into 100 parts by mass of an acidic solvent, then adding 2 to 6 parts by mass of an aldehyde monomer to the above acidic solvent, mixing uniformly, and reacting at −10° C. to 40° C. for 12 to 96 hours to obtain a halogenated benzene intermediate containing Tröger's bases; and
dispersing 1 to 10 parts by mass of the halogenated benzene intermediate containing Tröger's bases into 20 parts by mass of an aromatic hydrocarbon monomer, and under the condition of an inert gas, 0.03 to 0.06 parts by mass of a catalyst, 0.04 to 0.08 parts by mass of 1,1′-binaphthyl-2,2′-bisdiphenylphosphine, 0.8 to 1.2 parts by mass of sodium tert-butoxide and 0.8 to 1.6 parts by mass of benzophenone imine are added to the above aromatic hydrocarbon monomer, mixed uniformly, and reacted at 80° C. to 120° C. for 12 to 48 hours. After this, the reaction continues for 1 to 6 hours in the presence of a mixed solvents of 4 to 16 parts by mass of tetrahydrofuran and 10 to 40 parts by mass of 1 to 3 mol/L (e.g., 2M) inorganic acid, and finally the Tröger's base phenylenediamine monomer (PDA-TB) is obtained.

Optionally, the halogenated aniline monomer molecule is one or more selected from 4-fluoroaniline, 4-bromoaniline, 4-iodoaniline or 4-chloroaniline. Optionally, the aldehyde monomer is one or more selected from formaldehyde, paraformaldehyde, benzaldehyde, phenylacetaldehyde, phenylpropionaldehyde and octanal. Optionally, the aromatic hydrocarbon monomer is one or more selected from benzene, toluene, xylene or ethylbenzene. Optionally, the inert gas is at least one selected from nitrogen and argon. Optionally, the inorganic acid solvent is one selected from hydrochloric acid, sulfuric acid and nitric acid. Optionally, the catalyst is tris(dibenzylideneacetone) dipalladium.

According to an embodiment of the present disclosure, a method for preparing a highly selective acid-resistant polyamide nanofiltration membrane containing Tröger's base is provided, comprising the following steps:
preparing a Tröger's base phenylenediamine monomer as described above;
adding PDA-TB, polyethersulfone and a polar solvent to a round-bottomed flask, stirred regularly at a certain temperature, and then standing for defoaming to obtain a uniform and bubble-free casting solution;
coating the casting solution on a non-woven fabric and then placing into a coagulation bath aqueous solution for a non-solvent induced phase separation for 30 to 120 seconds, and after washing the surface with deionized water and drying at room temperature, the polyethersulfone support membrane with a surface rich in Tröger's base phenylenediamine monomer is obtained;
pouring a polyacyl chloride monomer solution on the surface of this polyethersulfone support membrane with PDA-TB and reacting for 30 to 300 seconds, and after removing the remaining organic phase solution and heat-treating at 25° C. to 40° C. for 20 to 60 minutes, the prepared membrane is immersed in deionized water for cleaning to obtain a highly selective acid-resistant polyamide nanofiltration membrane containing Tröger's bases.

Optionally, the polar solvent is one or more selected from N,N-dimethylformamide, N,N-dimethylacetamide, dimethyl sulfoxide or N-methylpyrrolidone. Optionally, the polyacyl chloride monomer is one or more selected from isophthaloyl chloride, biphenyl tetracarboxylic acid chloride, 1,3,5-triazine-2,4,6-triacyl chloride, adipoyl chloride, 4-biphenylcarbonyl chloride, trimesoyl chloride and phthaloyl chloride.

Optionally, in the static defoaming and the previous stirring steps, the stirring temperature is 20° C. to 60° C., the stirring time is 12 hours, and the static defoaming time is 12 to 24 hours.

Optionally, the content of the PDA-TB monomer is Owt % to 3 wt % when dispersing the Troger's base phenylenediamine monomer and the polyethersulfone in the polar solvent. Optionally, the weight percentages of the Tröger's base phenylenediamine monomer, the polyethersulfone and the polar solvent are calculated as 100% by weight:

| | |
|---|---|
| Tröger's base diamine monomer | 1 wt % to 3 wt %; |
| polyethersulfone | 10 wt % to 20 wt %; |
| polar solvent | balance. |

Optionally, the volume of the coagulation bath aqueous solution is 2 to 4 L, and the time of the non-solvent induced phase separation is 30 to 120 seconds.

Optionally, the time of drying at room temperature is 10 to 30 minutes.

Optionally, the concentration of the polyacyl chloride monomer is 0.01 wt % to 0.2 wt %.

Optionally, the organic solvent for dissolving the polyacyl chloride monomer is one or more selected from Isopar G, cyclohexane, n-nectane, n-hexane or n-heptane.

Optionally, the reaction time in the organic phase solution of the polyacyl chloride monomer is 50 to 200 seconds.

Optionally, the post-treatment temperature of the present invention is 25° C. to 35° C., and the time is 25 to 40 minutes.

Compared with the prior art, the highly selective acid-resistant polyamide nanofiltration membrane containing Tröger's base according to the method disclosed the present disclosure or prepared by the method has the following beneficial technical effects:

according to the method of the present disclosure, the synthesized Tröger's base phenylenediamine is added to the casting solution, and a highly selective acid-resistant polyamide nanofiltration membrane containing Tröger's base is prepared by coupling non-solvent induced phase separation and in-situ interfacial polymerization. Compared with the conventional poly(piperazine-amide) nanofiltration membrane, the full-Tröger's base polyamide segments contained in the nanofiltration membrane disclosed herein enables the pore size of the separation layer to be more uniform and the pore size distribution to be narrower; and enables the membrane surface to be electropositive under strong acidic conditions, which significantly improves the separation selectivity of the membrane for heavy metal ions or cationic dyes and H+ ions. Meanwhile, it gives the membrane excellent acid resistance, and thus the nanofiltration membrane has broad application prospects. In addition, according to the method in the present disclosure, the Tröger's base phenylenediamine monomer is directly enriched on the surface of the support membrane by a non-solvent induced phase separation method, which simplifies the preparation process of the nanofiltration membrane and improves the membrane production efficiency, thereby reducing the membrane production cost and facilitating industrialization.

### EXAMPLES

The technical solutions of the present disclosure are described in more detail below with reference to specific embodiments.

### Example 1

1. Synthesis of Tröger's base phenylenediamine monomer

At −15° C., 17.5 g of 4-bromoaniline, 6.2 g of paraformaldehyde and 200 mL of trifluoroacetic acid are added to a three-necked flask. The mixed solution was stirred to room temperature and continued to stir for 48 h. Slowly add excessive ice and ammonia water (25% mass concentration) to the mixed solution and stirred thoroughly. After being extracted three times with CH₂Cl₂, being dried over anhydrous MgSO₄ and adding silica gel and concentrate in vacuo, the residue was purified by alkaline alumina column chromatography (eluent:petroleum ether:ethyl acetate=10:1) to obtain the intermediate (2,8-dibromo-6H, 12H-5,11-methylenedibenzo[b,f][1,5]diazopyrimidine). Under the protection of nitrogen, 9.45 g of the obtained intermediate was dispersed into 200 mL of toluene. Subsequently, 0.6 g of 1,1′-binaphthyl-2,2′-bisdiphenylphosphine, 7.2 g of sodium tert-butoxide and 10 mL of benzophenone imine were added, and 0.3 g of tris(dibenzylideneacetone) dipalladium was also added as a catalyst, and then the reaction was refluxed for 24 hours. The obtained reaction mixture was concentrated in vacuo, and the residue was added to a mixed solution of 100 mL of tetrahydrofuran and 200 mL of hydrochloric acid (2M) and reacted for three hours. This reaction solution was poured into ice water and further was neutralized to alkaline by adding NH₃ (25%). After this, the mixture was extracted with CH₂Cl₂ for three times, washed with brine and dried with anhydrous MgSO₄, and then concentrated under vacuum by adding silica gel. The residue was subjected to alkaline alumina column chromatography (the eluent was dichloromethane:methanol=20:1) to obtain the Tröger's base phenylenediamine monomer (PDA-TB), dried in an oven at 60° C. for 36 h, and then stored in a sealed manner.

### 2. Preparation of Casting Solution

1 wt % of PDA-TB, 16.2 wt % of polyethersulfone E6020P (purchased from BASF GmbH) and dimethyl sulfoxide were added to a round-bottom flask, stirred at 25° C. for 12 hours and allowed to stand for 12 hours for defoaming, and finally a uniform bubble-free casting solution was obtained.

### 3. Non-Solvent Induced Phase Separation

The casting solution was uniformly poured onto the non-woven fabric S53 (purchased from HOKUETSU INDUSTRIES CO., LTD, Japan), then coated into a uniform liquid film by using a casting knife with 200 μm gap. The non-woven fabric coated with the liquid film was immersed into a 2 L coagulation bath aqueous solution for non-solvent induced phase separation for 60 seconds to obtain a PES porous support membrane with a surface rich in PDA-TB monomers.

### 4. In-Situ Interfacial Polymerization

A n-hexane solution of 0.075 w/v % 1,3,5-benzenetricarboxylic acid chloride was poured onto the surface of PES porous support membrane riched in PDA-TB monomer to perform the interfacial polymerization for 60 seconds. After removing the excess organic phase solution on the membrane surface and heat-treating at 30° C. for 30 minutes, a highly selective acid-resistant polyamide nanofiltration membrane containing Tröger's base was finally obtained.

### Example 2

The PDA-TB concentration in step 2 was changed from 1.0 wt % to 0.5 wt %, and other operations were the same as in Example 1. The performance data of the prepared membrane are listed in Table 1.

### Example 3

The PDA-TB concentration in step 2 was changed from 1.0 wt % to 1.5 wt %, and other operations were the same as in Example 1. The performance data of the prepared membrane are listed in Table 1.

### Example 4

The PDA-TB concentration in step 2 was changed from 1.0 wt % to 2.0 wt %, and other operations were the same as in Example 1. The performance data of the prepared membrane are listed in Table 1.

### Example 5

The concentration of 1,3,5-benzenetricarboxylic acid chloride in step 4 was changed from 0.075 w/v % to 0.025 w/v %, and other operations were the same as in Example 1. The performance data of the prepared membrane are listed in Table 1.

### Example 6

The concentration of 1,3,5-benzenetricarboxylic acid chloride in step 4 was changed from 0.075 w/v % to 0.05 w/v %, and other operations were the same as in Example 1. The performance data of the prepared membrane are listed in Table 1.

### Example 7

The concentration of 1,3,5-benzenetricarboxylic acid chloride in step 4 was changed from 0.075 w/v % to 0.1 w/v %, and other operations were the same as in Example 1. The performance data of the prepared membrane are listed in Table 1.

### Example 8

The concentration of 1,3,5-benzenetricarboxylic acid chloride in step 4 was changed from 0.075 w/v % to 0.125 w/v %, and other operations were the same as in Example 1. The performance data of the prepared membrane are listed in Table 1.

### Comparative Example 1

The Tröger's base phenylenediamine monomer PDA-TB in step 2 was replaced with piperazine, and other operations were the same as in Example 1. The prepared membrane was used for comparative testing, and the performance data are listed in Table 1-2.

TABLE 1
Comparison of Performance of the Nanofiltration
Membranes Prepared in Example 1-4

| Example | Water flux (L · m⁻² · h⁻¹) | Sodium sulfate rejection rate (%) |
|---|---|---|
| 1 | 36.89 | 98.47 |
| 2 | 90.20 | 93.75 |
| 3 | 19.64 | 96.16 |
| 4 | 30.09 | 94.69 |

TABLE 2
Comparison of Performance of Nanofiltration
Membrane Prepared in Example 5-8

| Example | Water flux (L · m⁻² · h⁻¹) | Sodium sulfate rejection rate (%) |
|---|---|---|
| 5 | 23.27 | 94.38 |
| 6 | 29.49 | 97.86 |
| 7 | 26.36 | 97.12 |
| 8 | 26.94 | 95.01 |

Example 1 and Comparative Example 1 were selected as examples to test the selective separation performance of iron ions and hydrogen ions. The separation test results of ferric chloride solution with pH=1 and 2000 ppm at 25° C. and a pressure of 0.6 MPa are shown in Table 3.

TABLE 3
Comparison of separation performance of iron ions
and hydrogen ions of the nanofiltration membranes
prepared in Example 1 and Comparative Example 1

| | | | Hydrogen | Selectivity |
|---|---|---|---|---|
| | | Iron ion | ion | of iron |
| | Water flux | rejection | rejection | ions and |
| | (L · m⁻² · h⁻¹) | rate (%) | rate (%) | hydrogen ions |
| Example 1 | 40.43 | 97.19 | 17.67 | 29.33 |
| Comparative | 76.13 | 95.72 | 24.00 | 17.57 |
| example 1 | | | | |

The separation test results for ferric sulfate at pH=1, 2000 ppm at 25° C. and a pressure of 0.6 MPa are shown in Table 4.

TABLE 4
Comparison of separation performance of iron ions
and hydrogen ions of the nanofiltration membranes
prepared in Example 1 and Comparative Example 1

| | | | Hydrogen | Selectivity |
|---|---|---|---|---|
| | | Iron ion | ion | of iron |
| | Water flux | rejection | rejection | ions and |
| | (L · m⁻² · h⁻¹) | rate (%) | rate (%) | hydrogen ions |
| Example 1 | 38.65 | 97.19 | 17.67 | 29.33 |
| Comparative | 80.41 | 95.72 | 24.00 | 17.57 |
| example 1 | | | | |

Example 1 and Comparative Example 1 were selected as examples to test the selective separation performance of iron ions and hydrogen ions. The separation test results of the crystal violet dye solution with pH=1 and 50 ppm at 25° C. and a pressure of 0.6 MPa are shown in Table 5.

TABLE 5
Comparison of Crystalline Violet Dye and Hydrogen
Ion Separation Performance of Nanofiltration Membrane
Prepared in Example 1 and Comparative Example

| | | Crystal | Hydrogen | Selectivity |
|---|---|---|---|---|
| | | violet | ion | of iron |
| | Water flux | rejection | rejection | ions and |
| | (L · m⁻² · h⁻¹) | rate (%) | rate (%) | hydrogen ions |
| Example 1 | 32.77 | 99.05 | 4.46 | 100.57 |
| Comparative | 89.68 | 97.93 | 4.51 | 46.13 |
| example 1 | | | | |

Example 1 and Comparative Example 1 were selected as examples to test the selective separation performance of iron ions and hydrogen ions. The separation test results of the rhodamine B dye solution with pH=1 and 50 ppm at 25° C. and a pressure of 0.6 MPa are shown in Table 6.

TABLE 6
Comparison of the separation performance of the rhodamine
B dye with hydrogen ions in the nanofiltration membranes
prepared in Example 1 and Comparative Example 1

| | | | | Selectivity |
|---|---|---|---|---|
| | | | Hydrogen | of iron |
| | | Rhodamine B | ion | ions and |
| | Water flux | rejection | rejection | hydrogen |
| | (L · m⁻² · h⁻¹) | rate (%) | rate (%) | ions |
| Example 1 | 27.86 | 99.52 | 7.34 | 193.04 |
| Comparative | 81.81 | 99.27 | 4.43 | 130.92 |
| example 1 | | | | |

Example 1 and Comparative Group 1 were selected as examples to test the selective separation performance of iron ions and hydrogen ions. The separation test results of the Congo red dye solution with pH=1 and 50 ppm at 25° C. and a pressure of 0.6 MPa are shown in Table 7.

TABLE 7
Comparison of separation performance of Congo
red dye and hydrogen ions in nanofiltration membranes
prepared in Example 1 and Comparative

| | | | Hydrogen | Selectivity |
|---|---|---|---|---|
| | | Congo red | ion | of iron |
| | Water flux | rejection | rejection | ions and |
| | (L · m⁻² · h⁻¹) | rate (%) | rate (%) | hydrogen ions |
| Example 1 | 30.56 | 99.28 | 5.26 | 131.58 |
| Comparative | 67.57 | 98.42 | 2.28 | 61.85 |
| example 1 | | | | |

Example 1 and Comparative Example 1 were selected as examples for testing the performance of acid resistance. The membranes prepared in Example 1 and Comparative Example 1 were immersed in 20 wt % sulfuric acid solution, and the separation test results for 2000 ppm sodium sulfate at 25° C. and 0.6 MPa were shown in Table 8 to Table 9:

TABLE 8
Changes in salt rejection of the nanofiltration
membranes prepared in Example 1 and Comparative
Example 1 after strong acid immersion

| Days | Example 1 | Comparative Example 1 |
|---|---|---|
| 0 | 97.68% | 93.75% |
| 3 | 97.15% | 60.88% |
| 6 | 95.65% | 65.84% |
| 9 | 95.25% | 25.01% |
| 12 | 94.10% | 18.01% |
| 15 | 93.14% | |
| 18 | 92.96% | |
| 24 | 92.60% | |
| 30 | 88.90% | |

TABLE 9
Changes in flux of the nanofiltration membranes prepared in Example
1 and Comparative Example 1 after strong acid immersion

| Days | Example 1 | Comparative Example 1 |
|---|---|---|
| 0 | 35.81 | 55.45 |
| 3 | 35.33 | 68.28 |
| 6 | 33.54 | 73.32 |
| 9 | 32.09 | 217.09 |
| 12 | 38.06 | 242.65 |
| 15 | 37.55 | |
| 18 | 39.08 | |
| 24 | 41.58 | |
| 30 | 46.46 | |

According to the above experimental results, it can be seen that the highly selective acid-resistant polyamide nanofiltration membrane containing Tröger's base prepared in the present disclosure has better acid resistance and separation selectivity than the traditional polypiperazineamide nanofiltration membrane. On day 12, the rejection performance of the poly(piperazine-amide) nanofiltration membrane decreased from 93.75% to 18.01%, and the polyamide separation layer was damaged by hydrogen ions, causing the entire membrane structure to be destroyed. The rejection performance of the polyamide nanofiltration membrane prepared with PDA-TB decreased from 97.86% to 88.90% after soaking in 20 wt % sulfuric acid solution for 30 days, which was significantly smaller than that of the traditional poly(piperazine-amide), and the membrane structure remained intact, indicating that the polyamide nanofiltration membrane containing Tröger's base has great potential in the application of acidic wastewater treatment. Under the strong acidic conditions of pH=1, the selective separation factor of the highly selective acid-resistant polyamide nanofiltration membrane containing Tröger's base for Fe³⁺/H⁺ is more than 29, which is much higher than the separation selectivity of the conventional poly(piperazine-amide) for Fe³⁺/H⁺ (about 17). Under the strong acidic condition of pH=1, the selective separation factors of the highly selective acid-resistant polyamide nanofiltration membrane containing Tröger's base for the two cationic dyes crystal violet (408 Da) and rhodamine B (479 Da)/H⁺ reach 100.57 and 193.04, respectively, which are much higher than the selective separation factors 46.13 and 130.92 of the conventional poly(piperazine-amide). Meanwhile, the selective separation factor of the highly selective acid-resistant polyamide nanofiltration membrane containing Tröger's base for the anionic dye Congo red/H⁺ (696 Da) exceeds 131.58, which is higher than the selective separation performance of the conventional poly(piperazine-amide) (about 61.85). These indicate the excellent performance and great industrial application potential of polyamide nanofiltration membrane containing Tröger's base in the treatment of acidic wastewater containing heavy metals or dyes.

What is claimed is:

1. A method for preparing a highly selective acid-resistant polyamide nanofiltration membrane containing Tröger's base, the method comprises:
preparing a Tröger's base phenylenediamine monomer;
dispersing the Troger's base phenylenediamine monomer and the polyethersulfone into a polar solvent, stirring uniformly, and then standing for defoaming to obtain a casting solution;
coating the casting solution on a non-woven fabric and immersing in a coagulation bath aqueous solution for a non-solvent induced phase separation, wherein a time for non-solvent induced phase separation is 30 to 120 seconds, then rinsing a membrane surface with deionized water, and finally drying the membrane at room temperature to obtain a polyethersulfone support membrane with a surface rich in Tröger's base phenylenediamine monomer; and
pouring the organic phase solution containing the polyacyl chloride monomer onto the surface of the polyethersulfone support membrane riched in Tröger's base phenylenediamine monomer, and after reacting for 30 to 300 seconds, removing the remaining organic phase solution and heat-treating at 25° C. to 40° C. for 20 to 60 minutes, the prepared membrane is immersed in deionized water for cleaning to finally obtain a highly selective acid-resistant polyamide nanofiltration membrane containing Tröger's bases.

2. The method according to claim 1, wherein the step of preparing the Tröger's base phenylenediamine monomer comprises:
dissolving 2 to 12 parts by mass of a halogenated aniline monomer molecule into 100 parts by mass of an acidic solvent, then adding 2 to 6 parts by mass of an aldehyde monomer, mixing uniformly, and reacting at −10° C. to 40° C. for 12 to 96 hours to obtain a halogenated benzene intermediate containing Tröger's bases; and
dispersing 1 to 10 parts by mass of the halogenated benzene intermediate containing Tröger's bases into 20 parts by mass of an aromatic hydrocarbon monomer, and under the condition of an inert gas, 0.03 to 0.06 parts by mass of a catalyst, 0.04 to 0.08 parts by mass of 1,1′-binaphthyl-2,2′-bisdiphenylphosphine, 0.8 to 1.2 parts by mass of sodium tert-butoxide and 0.8 to 1.6 parts by mass of benzophenone imine are added, mixed uniformly, and reacted at 80° C. to 120° C. for 12 to 48 hours, after this, the reaction continues for 1 to 6 hours at the presence of the mixed solvents of 4 to 16 parts by mass of tetrahydrofuran and 10 to 40 parts by mass of 1 to 3 mol/L inorganic acid, and finally the Tröger's base phenylenediamine monomer is obtained.

3. The method according to claim 2, wherein the halogenated aniline monomer molecule is one or more selected from 4-fluoroaniline, 4-bromoaniline, 4-iodoaniline and 4-chloroaniline.

4. The method according to claim 2, wherein the aldehyde monomer is one or selected from more formaldehyde, paraformaldehyde, benzaldehyde, phenylacetaldehyde, phenylpropionaldehyde and octanal.

5. The method according to claim 2, wherein the aromatic hydrocarbon monomer is one or more selected from benzene, toluene, xylene and ethylbenzene.

6. The method according to claim 2, wherein the inert gas is at least one selected from nitrogen and argon; and the inorganic acid is one selected from hydrochloric acid, sulfuric acid and nitric acid.

7. The method according to claim 2, wherein the catalyst is tris(dibenzylideneacetone) dipalladium.

8. The method according to claim 1, wherein the stirring is performed at a temperature of 20° C. to 60° C. for 12 to 30 hours, and then the defoaming is performed by standing for 12 to 24 hours.

9. The method according to claim 1, wherein the weight percentages of the Tröger's base phenylenediamine monomer, the polyethersulfone and the polar solvent are calculated as 100% by weight:

| | |
|---|---|
| Troger's base diamine monomer | 1 wt % to 3 wt %; |
| polyethersulfone | 10 wt % to 20 wt %; |
| polar solvent | balance. |

10. The method according to claim 1, wherein the polar solvent is one or more selected from N,N-dimethylformamide, N,N-dimethylacetamide or dimethyl sulfoxide, and N-methylpyrrolidone.

11. The method according to claim 9, wherein the volume of the coagulation bath aqueous solution is 2 to 4 L, and the time of the non-solvent induced phase separation is 45 to 80 seconds.

12. The method according to claim 9, wherein the time of drying at room temperature is 10 to 30 minutes.

13. The method according to claim 1, wherein the polyacyl chloride monomer is one or more selected from isophthaloyl chloride, 1,3,5-triazine-2,4,6-triacyl chloride, adipoyl chloride, 4-biphenylcarbonyl chloride, trimesoyl chloride and phthaloyl chloride.

14. The method according to claim 13, wherein the concentration of the polyacyl chloride monomer in the organic phase solution containing the polyacyl chloride monomer is 0.01 wt % to 0.2 wt %; the organic solvent in the organic phase solution containing the polyacyl chloride monomer is one or more selected from Isopar G, cyclohexane, n-alkane, n-hexane and n-heptane.

15. A highly selective acid-resistant polyamide nanofiltration membrane containing Tröger's base prepared by the preparation method according to claim 1.
